(19) United States
(12) Patent Application Publication
(10) Pub. No.: US 2026/0217827 A1
(43) Pub. Date: Jul. 30, 2026

(54) METHODS FOR TREATING RENAL CELL CARCINOMA

(71) Applicant: Corvus Pharmaceuticals, Inc., South San Francisco, CA (US)

(72) Inventors: Richard A. Miller, South San Francisco, CA (US); Lih-Yun Hsu, South San Francisco, CA (US)

(21) Appl. No.: 19/142,365

(22) PCT Filed: Dec. 26, 2023
(86) PCT No.: PCT/US2023/085895

§ 371 Date: Jun. 23, 2025

Related U.S. Application Data
(60) Provisional application No. 63/477,256, filed on Dec. 27, 2022.

(51) Int. Cl. C07K 16/28; A61K 31/519; A61K 39/00; A61P 35/00; C12Q 1/6809; C12Q 1/6886

(52) U.S. Cl. CPC C07K 16/2818; A61K 31/519; A61P 35/00; C12Q 1/6809; C12Q 1/6886; A61K 2039/505; A61K 2039/545; C12Q 2600/106; C12Q 2600/158

(57) ABSTRACT

Provided herein, inter alia, are methods of treating renal cell carcinoma with an adenosine A2A receptor antagonist and a CTLA-4 inhibitor. The method may optionally further comprise administering a PD-1 inhibitor.

### CROSS-REFERENCE TO RELATED APPLICATIONS

This application claims the benefit of priority to U.S. Application No. 63/477,256 filed Dec. 27, 2022, the disclosure of which is incorporated by reference herein.

### BACKGROUND

Renal cell carcinoma (also known as kidney cancer) is a cancer that originates in the kidneys. Renal cell carcinoma can progressively develop into widespread metastatic disease. Originating on the surface of the renal cortex, the small, localized primary tumor rarely produces noticeable symptoms during the early stages of disease (e.g., Stages I and II). Early stage renal cell carcinoma is frequently identified incidentally through diagnostic readings performed in the evaluation of an unrelated condition (MRI scans, for example). As the disease progresses, symptoms can present as a classic triad consisting of hematuria, a palpable mass in the flank or abdomen, and pain. In Stages I-III, partial (e.g., Stage I) or total (Stages II and III) surgical removal of the kidney remains the only known effective therapy for localized renal cell carcinoma. In 30% to 50% of patients, progression to metastatic disease (Stage IV) occurs prior to initial diagnosis. However, traditional therapeutic modalities, including chemotherapy and radiation, are largely ineffective in Stage IV patients. There is a need in the art for new and effective treatments for renal cell carcinoma. The disclosure is directed to this, as well as other, important ends.

### BRIEF SUMMARY

Provided herein is a method for treating renal cell carcinoma in a patient in need thereof, the method comprising: (a) detecting an increased level of gene expression of a biomarker gene, relative to a control, in a biological sample obtained from the patient, wherein the biomarker gene is CXCL1, CXCL2, CXCL3, CXCL5, CXCL6, CXCL8, IL-1β, PTGS2, or a combination of two or more thereof; and (b) administering to the patient an effective amount of an adenosine A2A receptor antagonist and an effective amount of a CTLA-4 inhibitor. The combination of the CTLA-4 inhibitor and adenosine A2A receptor antagonist work in concert to eliminate and block immunosuppressive T regs and myeloid suppressor cells.

Provided herein is a method for treating renal cell carcinoma in a patient in need thereof, the method comprising administering to the patient an effective amount of an adenosine A2A receptor antagonist and an effective amount of a CTLA-4 inhibitor; wherein a biological sample obtained from the patient has an increased level of gene expression of a biomarker gene, relative to a control, wherein the biomarker gene is CXCL1, CXCL2, CXCL3, CXCL5, CXCL6, CXCL8, IL-1β, PTGS2, or a combination of two or more thereof.

Provided herein is a method for treating renal cell carcinoma in a patient in need thereof, the method comprising: (a) detecting an increased level of myeloid suppressor cells relative to a control in a biological sample obtained from the patient; and (b) administering to the patient an effective amount of an adenosine A2A receptor antagonist and an effective amount of a CTLA-4 inhibitor. The combination of the CTLA-4 inhibitor and adenosine A2A receptor antagonist work in concert to eliminate and block immunosuppressive T regs and myeloid suppressor cells.

Provided herein is a method for treating renal cell carcinoma in a patient in need thereof, the method comprising administering to the patient an effective amount of an adenosine A2A receptor antagonist and a CTLA-4 inhibitor; wherein a biological sample obtained from the patient has an increased level of myeloid suppressor cells relative to a control.

Provided herein is a method for treating renal cell carcinoma in a patient in need thereof comprising administering to the patient an effective amount of an adenosine A2A receptor antagonist, an effective amount of a CTLA-4 inhibitor, and an effective amount of a PD-1 inhibitor, wherein the effective amount of the CTLA-4 inhibitor and/or the PD-1 inhibitor is a reduced effective amount.

These and other embodiments of the disclosure are described herein.

### BRIEF DESCRIPTION OF THE DRAWINGS

FIGS. 1A-1E show that the combination of ciforadenant and a CTLA-4 inhibitor (anti-CTLA-4 antibody) promotes superior production of pro-inflammatory cytokines when compared to either agent alone, including IFN-gamma (FIG. 1A), TNF-alpha (FIG. 1B), IL-6 (FIG. 1C), IL-12/IL-23 p40 (FIG. 1D), and CXCL10 (FIG. 1E). Mouse tumors from different treatment groups were lysed, then the proteins were extracted for cytokine measurements.

FIGS. 2A-2C show that the triple combination of ciforadenant, CTLA-4 inhibitor (anti-CTLA-4 antibody), and PD-1 inhibitor (anti-PD-1 antibody) triggers activation of IL-12/STAT4 signaling axis (FIG. 2A), resulting in skewing into Th1 T cells (FIG. 2B) and reduced exhaustion in CD8 T cells (FIG. 2C). Transcript levels of the indicated genes in the IL-12/STAT4 axis were identified using a Nanostring™ mouse myeloid panel and analyzed on a nCounter® MAX Analysis System. The production of Th1 effector cytokines marked by IFNγ and TNFα from CD4 T cells and exhausted CD8 T cells marked by Eomes (Eomesodermin) and LAG3 (Lymphocyte Activating Gene 3) from mouse tumors were detected by flow cytometry.

FIG. 3 shows that the ciforadenant response is high when the adenosine signature is high (e.g., elevated level of gene expression for CXCL1, CXCL2, CXCL3, CXCL5, CXCL6, CXCL8, IL-1β, and PTGS2). Note that CXCL8 is equivalent to IL-8.

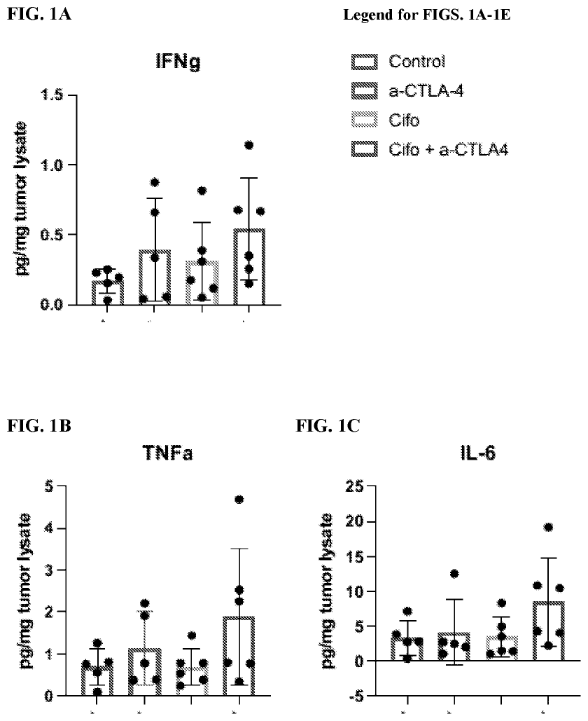

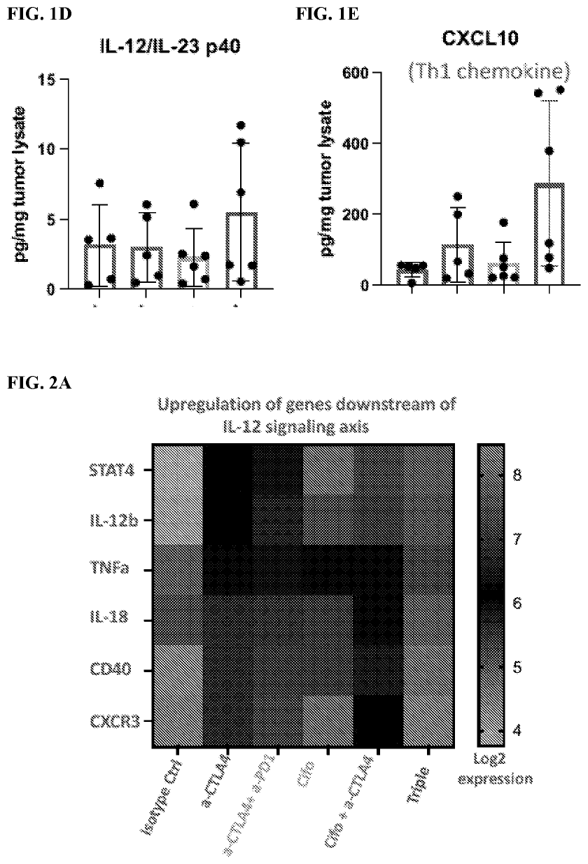

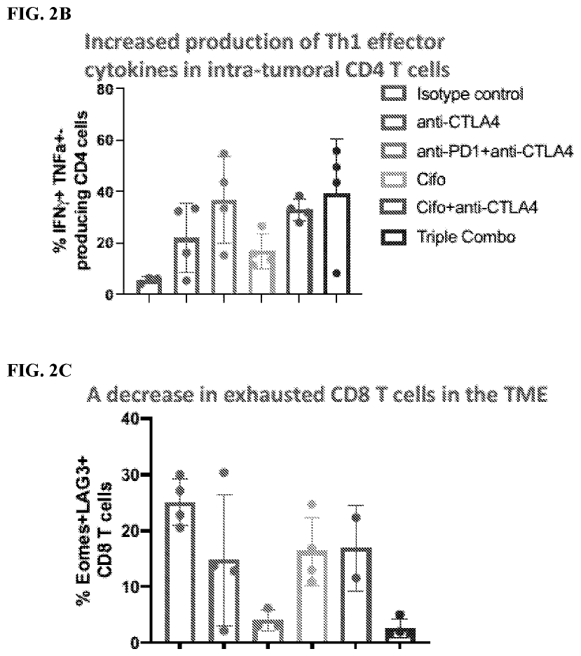

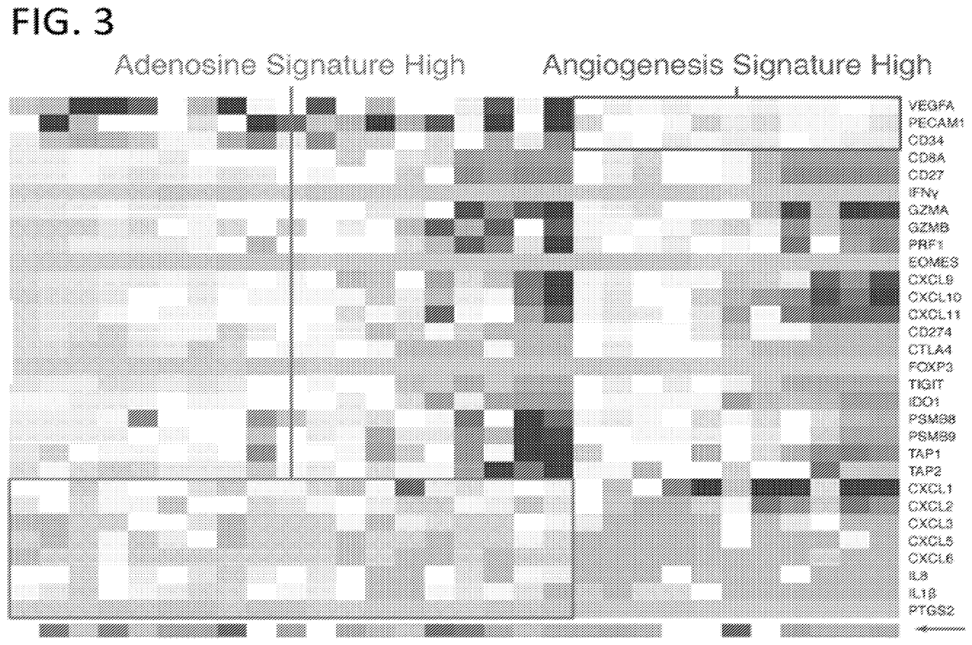

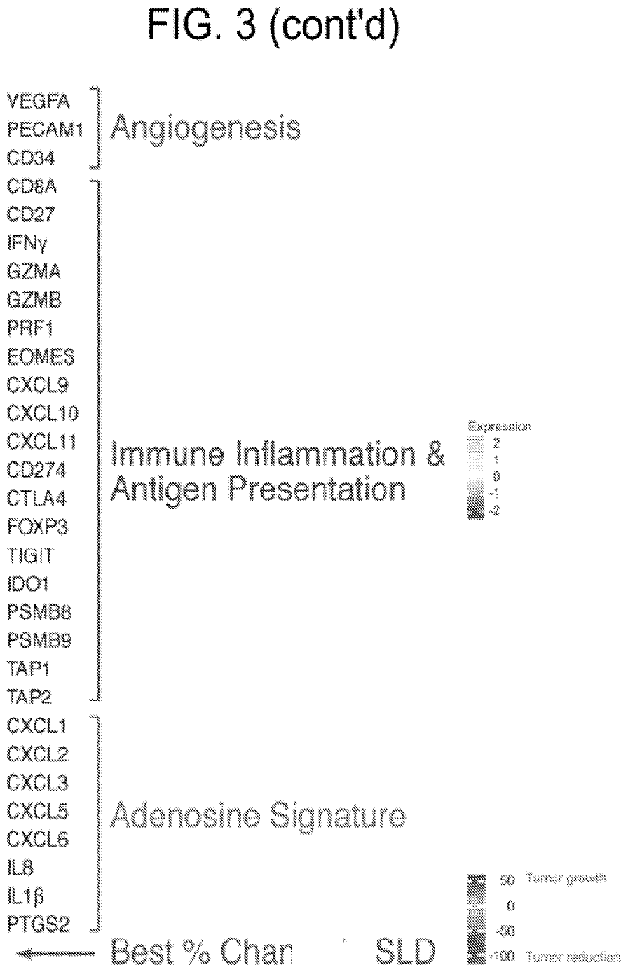

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

As defined herein, the term “inhibition,” “inhibit,” “inhibiting” and the like in reference to a protein-inhibitor interaction (e.g. pathway inhibition) means negatively affecting (e.g. decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor” or “antagonist” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In embodiments, expression or activity is 1.5-fold, 2-fold, 3-fold, 4-fold, 5-fold, 10-fold or lower than the expression or activity in the absence of the antagonist.

The term “adenosine A2A receptor” or “A2A adenosine receptor” as provided herein includes any of the recombinant or naturally-occurring forms of the adenosine A2A receptor (ADORA2A) or variants or homologs thereof that maintain ADORA2A protein activity (e.g. within at least 50%, 80%, 90%, 95%, 96%, 97%, 98%, 99% or 100% activity compared to ADORA2A). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ADORA2A polypeptide. In embodiments, ADORA2A is the protein as identified by the NCBI sequence reference GI:5921992, homolog or functional fragment thereof.

“Adenosine pathway inhibitor” refers to a molecule that inhibits the activity of the adenosine pathway. An adenosine pathway inhibitor may be, without limitation, an adenosine receptor (e.g., adenosine A2A receptor or adenosine A2B receptor) antagonist, a CD73 antagonist, a CD38 antagonist, a CD39 antagonist, or adenosine deaminase. Examples of CD73 antagonists can be found in WO 2017/100670, WO 2018/013611, and WO 2018/187512, each of which is incorporated herein by reference in its entirety.

“Adenosine receptor antagonist” refers to a molecule that inhibits activity of adenosine receptors (e.g. A2A or A2B receptors), typically through direct action. Adenosine receptors antagonists can be small or large molecule antagonists. In embodiments, the adenosine A2A receptor antagonist is ciforadenant.

The term “CTLA-4” or “cytotoxic T-lymphocyte protein 4” as provided herein includes any of the recombinant or naturally-occurring forms of the CTLA-4 protein receptor or variants or homologs thereof that maintain CTLA-4 protein activity (e.g. within at least 50%, 80%, 90%, 95%, 96%, 97%, 98%, 99% or 100% activity compared to CTLA-4). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, CTLA-4 is the protein as identified by UniProt number P16410, a homolog, or a functional fragment thereof.

CTLA-4 is expressed in immunosuppressive T regulatory cells and, together with myeloid suppressor cells, further dampen immune responses to tumors. Anti-CTLA4 antibodies bind to CTLA-4 positive T regs and cause their elimination.

A “CTLA-4 inhibitor” as provided herein refers to a substance capable of detectably lowering expression of or activity level of CTLA-4 compared to a control. The inhibited expression or activity of CTLA-4 can be 10%, 20%, 30%, 40%, 50%, 60%, 70%, 80%, 90% or less than that in a control. In embodiments, the inhibition is 1.5-fold, 2-fold, 3-fold, 4-fold, 5-fold, 10-fold, or more in comparison to a control. An “inhibitor” is a compound or small molecule that inhibits CTLA-4, e.g., by binding, partially or totally blocking stimulation of the CTLA-4 receptor, decrease, prevent, or delay activation of the CTLA-4 protein, or inactivate, desensitize, or down-regulate signal transduction, gene expression or enzymatic activity of the CTLA-4 protein. In embodiments, the CTLA-4 inhibitor inhibits CTLA-4 activity or expression. In embodiments, the CTLA-4 inhibitor is a compound or a small molecule. In embodiments, the CTLA-4 inhibitor is an antibody. In embodiments, the CTLA-4 inhibitor is ipilimumab.

A “PD-1 protein” or “PD-1” as referred to herein includes any of the recombinant or naturally-occurring forms of the programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1 protein” or “PD-L1” as referred to herein includes any of the recombinant or naturally-occurring forms of the programmed death-ligand 1 (PD-L1) also known as cluster of differentiation 274 (CD 274) or variants or homologs thereof that maintain PD-L1 protein activity (e.g. within at least 50%, 80%, 90%, 95%, 96%, 97%, 98%, 99% or 100% activity compared to PD-L 1 protein). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto. In embodiments, the PD-L1 protein is substantially identical to the protein identified by the UniProt reference number Q9EP73 or a variant or homolog having substantial identity thereto.

“PD-1 inhibitor” as provided herein refers to a substance capable of detectably lowering expression of or activity level of the PD-1 signaling pathway compared to a control. The inhibited expression or activity of the PD-1 signaling pathway can be 10%, 20%, 30%, 40%, 50%, 60%, 70%, 80%, 90% or less than that in a control. In embodiments, the inhibition is 1.5-fold, 2-fold, 3-fold, 4-fold, 5-fold, 10-fold, or more in comparison to a control. An “inhibitor” is a compound or small molecule that inhibits the PD-1 signaling pathway e.g., by binding, partially or totally blocking stimulation of the PD-1 pathway, decrease, prevent, or delay activation of the PD-1 pathway, or inactivate, desensitize, or down-regulate signal transduction, gene expression or enzymatic activity of the PD-1 pathway. In embodiments, the PD-1 inhibitor inhibits PD-1 activity or expression. In embodiments, the PD-1 inhibitor” is a compound or a small molecule. In embodiments, the PD-1 inhibitor is an antibody. In embodiments, the PD-1 inhibitor is nivolumab.

The term “myeloid cells” refers to granulocytes and monocytes that are differentiated descendants from common progenitors derived from hematopoietic stem cells in the bone marrow. Commitment to either granulocytes or monocytes is controlled by transcription factors followed by terminal differentiation in response to specific colony-stimulating factors and release into the circulation.

The term “myeloid suppressor cells” or “myeloid-derived suppressor cells” a heterogeneous population of cells of myeloid origin that expand during various pathological conditions (including cancer, inflammation, and trauma), and are characterized by the increased production of reactive oxygen and nitrogen species, and by arginase 1 activity. Myeloid suppressor cells suppress T-cell responses to pathological conditions, such as cancer. Myeloid suppressor cells can be measured by methods known in the art, such as those described, for example, by Florcken et al, Immunol Lett, 168 (2):260-267 (2015).

“Nucleic acid” refers to nucleotides (e.g., deoxyribonucleotides or ribonucleotides) and polymers thereof in either single-, double-or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a poly peptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The term “messenger RNA” or “mRNA” refer a single-stranded molecule of RNA that corresponds to the genetic sequence of a gene, and is read by a ribosome in the process of synthesizing a protein.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The phrase “specifically (or selectively) binds” to an antibody or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. An RNA that is the predominant species present in a preparation is substantially purified.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acids or polypeptide sequences refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/ or the like). Such sequences are then the to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence. The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “about” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value. In embodiments, “about” includes the specified value.

The singular terms “a,” “an,” and “the” include the plural reference unless the context clearly indicates otherwise.

A “therapeutic agent” as used herein refer to an agent (e.g., compound, biologic, pharmaceutical composition) that when administered to a subject will have the intended prophylactic effect, e.g., treating renal cell carcinoma, or their symptoms or the intended therapeutic effect, including any objective or subjective parameter of treatment such as abatement; remission; diminishing of symptoms; slowing in the rate of degeneration or decline; making the final point of degeneration less debilitating; or improving a patient's physical or mental well-being. Adenosine A2A inhibitors, CTLA-4 inhibitors, and PD-1 inhibitors are examples of a “therapeutic agent.”

“Treating” or “treatment” as used herein and as well-understood in the art includes any approach for obtaining beneficial clinical results for a patient. Beneficial clinical results includes, but is not limited to, alleviation or amelioration of one or more symptoms of a disease (i.e., renal cell carcinoma), diminishment of the extent of the disease, stabilizing (i.e., not worsening) the disease, delaying or slowing progression of the disease, amelioration or palliation of the disease, and remission, whether partial or total and whether detectable or undetectable. Treatment may relieve the disease's symptoms, fully or partially remove the disease's underlying cause, shorten a disease's duration, or do a combination of these things. Treatment methods include administering to a subject a therapeutically effective amount of a therapeutic agent. The administering step may comprise a single administration or a series of administrations. The length of the treatment period depends on a variety of factors, such as the severity of the condition, the age of the patient, the concentration of therapeutic agent, the activity of the compositions used in the treatment, or a combination thereof. It will also be appreciated that the effective dosage of therapeutic agent used for the treatment may increase or decrease over the course of a particular treatment regime. Changes in dosage may result and become apparent by standard diagnostic assays known in the art. Treating does not include preventing.

In embodiments, “treating renal cell carcinoma” means preventing an increase in size or volume of the cancer tumor. In embodiments, treating renal cell carcinoma includes decreasing the size of volume of the cancer tumor. In embodiments, treating renal cell carcinoma includes eliminating the cancer tumor altogether. In embodiments, a cancer tumor is eliminated when it is not detectable by an imaging test such as magnetic resonance imaging (MRI), a positron emission tomography (PET) scan, X-ray computed tomography (CT), ultrasound, or single-photon emission computed tomography (SPECT). In embodiments, treating renal cell carcinoma means reducing or preventing metastasis of the cancer tumor.

“Patient” or “subject in need thereof” refers to a living organism suffering from renal cell carcinoma that can be treated by administration of a therapeutic agent as provided herein. Non-limiting examples include humans and other mammals, such as dogs and cats. In embodiments, a patient is human.

The term “therapeutically effective amount” and “effective amount” as used herein refer to the amount of therapeutic agent sufficient to treat renal cell carcinoma. For any therapeutic agent described herein, the therapeutically effective amount can be initially determined from cell culture assays. Target concentrations will be those concentrations of therapeutic agent that are capable of achieving the methods described herein, as measured using the methods described herein or known in the art. As is known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Dosages may be varied depending upon the requirements of the patient and the therapeutic agent being employed. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state. A “therapeutically effective amount” can also be found on the label or Prescribing Information for commercially available therapeutic agents. In embodiments, the “effective amount” of ciforadenant is about 200 mg per day. In embodiments, the “effective amount” of ciforadenant is about 100 mg twice per day. In embodiments, the “effective amount” of ipilimumab is about 1 mg/kg about once every three weeks. In embodiments, the “effective amount” of nivolumab is about 3 mg/kg about once every three weeks.

The term “reduced effective amount” refers to an amount that is less than an effective amount when compared to the amount that is normally administered for the treatment of renal cell carcinoma. The reduced effective amount is the same dose administered less frequently, a lower dose administered with the same frequency as the normal dose, or a lower dose administered less frequently. In embodiments, the reduced effective amount is the same dose administered less frequently. In embodiments, the reduced effective amount is a lower dose administered with the same frequency as the normal dose. In embodiments, the reduced effective amount is a lower dose administered less frequently. As described herein, a “reduced effective amount” of a CTLA-4 inhibitor (ipilimumab) and/or a PD-1 inhibitor (nivolumab) is effective to treat renal cell carcinoma when the CTLA-4 inhibitor (ipilimumab), PD-1 inhibitor (nivolumab), and adenosine A2A receptor antagonist (ciforadenant) are administered as a combination therapy for treating renal cell carcinoma.

The term “administering” is used in accordance with its plain and ordinary meaning and includes oral, topical, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, or transdermal). Parenteral administration includes, e.g., intravenous, intramuscular, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any therapeutic agent other than the recited therapeutic agent.

“Biological sample” or “sample” refer to materials obtained from or derived from a subject or patient. A biological sample includes sections of tissues such as biopsy. Such samples include bodily fluids such as blood and blood fractions or products (e.g., serum, plasma, platelets, red blood cells, and the like), sputum, tissue, cultured cells (e.g., primary cultures, explants, and transformed cells), stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. In embodiments, a biological sample is blood. In embodiments, a biological sample is a peripheral blood sample. In embodiments, a biological sample is a serum sample (e.g., the fluid and solute component of blood without the clotting factors). In embodiments, a biological sample is a plasma sample (e. g, the liquid portion of blood). In embodiments, a biological sample is a tumor sample. In embodiments, a biological sample is a primary tumor sample. In embodiments, a biological sample is a metastatic tumor sample. In embodiments, a biological sample is a resected tumor sample. In embodiments, a biological sample is a tumor biopsy sample. In embodiments, a biological sample is a resected tumor sample from a primary tumor. In embodiments, a biological sample is a resected tumor sample from a metastisic tumor. In embodiments, a biological sample is a tumor biopsy sample from a primary tumor. In embodiments, a biological sample is a tumor biopsy sample from a metastisic tumor. In embodiments, a tumor sample is tumor cells. Biological samples can be taken from a subject by methods known in the art and can be analyzed by methods known in the art.

The term “biomarker” refers to an indicator, e.g., a predictive, prognostic, and/or a pharmacodynamic indicator, which can be detected in a biological sample. The biomarker may serve as an indicator of the likelihood a patient will respond to a particular therapeutic treatment or a particular subtype of a disease or disorder, characterized by certain molecular, pathological, histological, and/or clinical features. In embodiments, a biomarker is a gene or a set of genes (i.e., a biomarker gene). Biomarkers include, but are not limited to, polynucleotides (e.g., DNA, and/or RNA), polynucleotide copy number alterations (e.g., DNA copy numbers), polypeptides, or polypeptide and polynucleotide modifications (e.g., posttranslational modifications). In embodiments, the biomarker gene is CXCL1, CXCL2, CXCL3, CXCL5, CXCL6, CXCL8, IL-1β, PTGS2, or a combination of two or more thereof.

The terms “expression level,” “amount,” or “level” of a biomarker is a detectable level in a biological sample. “Expression” generally refers to the process by which information (e.g., gene-encoded and/or epigenetic) is converted into the structures present and operating in the cell. Therefore, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer RNA, ribosomal RNA, non-coding RNA). Expression levels can be measured by methods known to one skilled in the art and also disclosed herein. The expression level or amount of a biomarker can be used to identify/characterize a subject who may likely respond to, or benefit from, a particular therapeutic agent (e.g., an adenosine A2A receptor antagonist, a CTLA-4 inhibitor, a PD-1 inhibitor).

The terms an “elevated expression level” or “elevated level” of gene expression is an expression level of the gene that is higher than the expression level of the gene in a control. The control may be any control known in the art, such as those described herein. In embodiments, an “elevated expression level” of the biomarker gene compared to the control (when the expression level of the biomarker is greater than the corresponding control) is, for example, an increase in the expression level of about 10%, 20%, 30%, 40%, 50%, 60%, 70%, 80%, 90%, 95%, 96%, 97%, 98% or 99% or greater relative to the control. In embodiments, an “elevated expression level” of the biomarker gene is an amount that is statistically significantly greater than the expression level of the control.

Biomarker levels may be detected at either the protein or gene expression level. Proteins expressed by biomarkers can be quantified by immunohistochemistry (IHC) or flow cytometry with an antibody that detects the proteins. Biomarker expression can be and quantified by multiple platforms known in the art. Quantifying biomarker (gene) expression can alternatively be referred to as detecting a level of biomarker (gene) expression. Platforms that can be used to quantify biomarker (gene) expression or detect levels of biomarker (gene) expression include quantitative polymerase chain reaction (qPCR), multiplex quantitative polymerase chain reaction (multiplex qPCR), real-time polymerase chain reaction (rtPCR), Nanostring (e.g., an amplification-free technology that measures nucleic acid content by counting molecules directly), RNA-sequencing (using next-generation sequencing (NGS) to reveal the presence and quantity of RNA in a biological sample), or in situ hybridization. There is a range of biomarker expression across as measured by Nanostring. In embodiments, quantitative rtPCR, Nanostring, RNA-sequencing (RNAseq), and in situ hybridization are used to quantitate biomarker gene expression. In embodiments, biomarker expression is quantified by RNAseq. In embodiments, biomarker expression is quantified by multiplex qPCR. In embodiments, biomarker expression is quantified by NanoString. For Nanostring, RNA is extracted from a biological sample and a known quantity of RNA is placed on the Nanostring machine for gene expression detection using gene specific probes. The number of counts of biomarkers within a sample is determined and normalized to a set of housekeeping genes. To determine a threshold for increased or decreased biomarker levels, one skilled in the art could assess biomarker levels in a control group of samples and select the 10^{th}, 20^{th}, 25^{th}, 30^{th}, 40^{th}, 50^{th}, 60^{th}, 70^{th}, 75^{th}, 80^{th} or 90^{th} percentile of biomarker gene expression. In embodiments, the increased or decreased expression of biomarkers may be determined by calculating the H-score for the expression of the biomarkers. Thus, the increased or decreased expression of biomarkers may have an H-score. As used herein, an “H-score” or “Histoscore” is a numerical value determined by a semi-quantitative method commonly known for immunohistochemically evaluating protein expression in tumor samples.

“Control” is used in accordance with its plain ordinary meaning and refers to an assay, comparison, or experiment in which the subjects or reagents of the experiment are treated as in a parallel experiment except for omission of a procedure, reagent, or variable of the experiment. In embodiments, the control is used as a standard of comparison in evaluating experimental effects. In embodiments, the control is a gene expression level against which another gene expression level (e.g. the gene expression level of a biomarker gene disclosed herein) is compared (e.g., to make a diagnostic (e.g., predictive and/or prognostic) and/or therapeutic determination. In embodiments, a control is a healthy patient or a healthy population of patients. In embodiments, a healthy patient is a patient that does not have renal cell carcinoma. In embodiments, the control is an average value from population of healthy patients. In embodiments, the control is a level of expression of the biomarker gene that has been correlated with the responsiveness/non-responsiveness to a particular therapeutic agent. In embodiments, a control is a pre-assigned value, e.g., a cut-off value which was previously determined to significantly separate a first group of patients (e.g., patients with renal cell carcinoma) from a second group of patients (e.g., healthy patients). In embodiments, the cut-off value is the median or mean (preferably median) gene expression level in the reference population. A control can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of expression of a gene/protein or a subset of genes/proteins, a control comprises the average amount of expression (e.g., protein or mRNA) in a population of subjects (e.g., with renal cell carcinoma) or in a healthy or general population. In embodiments, the control comprises an average amount (e.g. amount of expression) in a population in which the number of subjects (n) is 5 or more, 20 or more, 50 or more, 100 or more, 1,000 or more, and the like. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. Other controls can be used in the methods described herein in order to confirm the accuracy of results and to confirm the absence of impurities or contamination of reagents.

### Methods

Provided herein is a method for treating renal cell carcinoma in a patient in need thereof comprising: (a) detecting an increased level of gene expression, relative to a control, of a biomarker gene in a biological sample obtained from the patient, wherein the biomarker gene is CXCL1, CXCL2, CXCL3, CXCL5, CXCL6, CXCL8, IL-1β, PTGS2, or a combination of two or more thereof; and (b) administering to the patient an effective amount of an adenosine A2A receptor antagonist and an effective amount of a CTLA-4 inhibitor. In embodiments, the adenosine A2A receptor antagonist is ciforadenant. In embodiments, the CTLA-4 inhibitor is ipilimumab. In embodiments, the method further comprises administering to the patient an effective amount of a PD-1 inhibitor. In embodiments, the PD-1 inhibitor is nivolumab. In embodiments, the biological sample is blood, tumor tissue, or tumor cells. In embodiments, the biological sample is tumor tissue or tumor cells. In embodiments, the biological sample is tumor tissue. In embodiments, the biological sample is tumor cells. In embodiments, the control is a population of healthy subjects (e.g., that do not have renal cell carcinoma).

Provided herein is a method for treating renal cell carcinoma in a patient in need thereof comprising: (a) detecting an increased level of gene expression, relative to a control, of a biomarker gene in a biological sample obtained from the patient, wherein the biomarker gene is CXCL1, CXCL2, CXCL3, CXCL5, CXCL6, CXCL8, IL-1β, PTGS2, or a combination of two or more thereof; and (b) administering to the patient an effective amount of ciforadenant and an effective amount of ipilimumab. In embodiments, the effective amount of ciforadenant is about 200 mg per day and the effective amount of ipilimumab is about 1 mg/kg about once every three weeks. In embodiments, the effective amount of ciforadenant is about 100 mg twice per day and the effective amount of ipilimumab is about 1 mg/kg about once every three weeks. In embodiments, the biological sample is blood, tumor tissue, or tumor cells. In embodiments, the biological sample is tumor tissue or tumor cells. In embodiments, the biological sample is tumor tissue. In embodiments, the biological sample is tumor cells. In embodiments, the control is a population of healthy subjects (e.g., that do not have renal cell carcinoma).

Provided herein is a method for treating renal cell carcinoma in a patient in need thereof, the method comprising: (a) detecting an increased level of gene expression, relative to a control, of a biomarker gene in a biological sample obtained from the patient, wherein the biomarker gene is CXCL1, CXCL2, CXCL3, CXCL5, CXCL6, CXCL8, IL-1β, PTGS2, or a combination of two or more thereof; and (b) administering to the patient an effective amount of ciforadenant, an effective amount of ipilimumab, and an effective amount of nivolumab. In embodiments, the effective amount of ciforadenant is about 200 mg per day, the effective amount of ipilimumab is about 1 mg/kg about once every three weeks, and the effective amount of nivolumab is about 3 mg/kg about once every three weeks. In embodiments, the effective amount of ciforadenant is about 100 mg twice per day, the effective amount of ipilimumab is about 1 mg/kg about once every three weeks, and the effective amount of nivolumab is about 3 mg/kg about once every three weeks. In embodiments, the biological sample is blood, tumor tissue, or tumor cells. In embodiments, the biological sample is tumor tissue or tumor cells. In embodiments, the biological sample is tumor tissue. In embodiments, the biological sample is tumor cells. In embodiments, the control is a population of healthy subjects (e.g., that do not have renal cell carcinoma). In embodiments, the effective amount of ipilimumab and/or the effective amount of nivolumab is a reduced effective amount.

Provided herein is a method for treating renal cell carcinoma in a patient in need thereof, the method comprising administering to the patient an effective amount of an adenosine A2A receptor antagonist and an effective amount of a CTLA- 4 inhibitor; wherein a biological sample obtained from the patient has an increased level of gene expression of a biomarker gene relative to a control, wherein the biomarker gene is CXCL1, CXCL2, CXCL3, CXCL5, CXCL6, CXCL8, IL-1β, PTGS2, or a combination of two or more thereof. In embodiments, the adenosine A2A receptor antagonist is ciforadenant. In embodiments, the CTLA-4 inhibitor is ipilimumab. In embodiments, the method further comprises administering to the patient an effective amount of a PD-1 inhibitor. In embodiments, the PD-1 inhibitor is nivolumab. In embodiments, the biological sample is blood, tumor tissue, or tumor cells. In embodiments, the biological sample is tumor tissue or tumor cells. In embodiments, the biological sample is tumor tissue. In embodiments, the biological sample is tumor cells. In embodiments, the control is a population of healthy subjects (e.g., that do not have renal cell carcinoma).

Provided herein is a method for treating renal cell carcinoma in a patient in need thereof, the method comprising administering to the patient an effective amount of ciforadenant and an effective amount of ipilimumab, wherein a biological sample obtained from the patient has an increased level of gene expression of a biomarker gene relative to a control, wherein the biomarker gene is CXCL1, CXCL2, CXCL3, CXCL5, CXCL6, CXCL8, IL-1β, PTGS2, or a combination of two or more thereof. In embodiments, the effective amount of ciforadenant is about 200 mg per day and the effective amount of ipilimumab is about 1 mg/kg about once every three weeks. In embodiments, the effective amount of ciforadenant is about 100 mg twice per day and the effective amount of ipilimumab is about 1 mg/kg about once every three weeks. In embodiments, the biological sample is blood, tumor tissue, or tumor cells. In embodiments, the biological sample is tumor tissue or tumor cells. In embodiments, the biological sample is tumor tissue. In embodiments, the biological sample is tumor cells. In embodiments, the control is a population of healthy subjects (e.g., that do not have renal cell carcinoma).

Provided herein is a method for treating renal cell carcinoma in a patient in need thereof, the method comprising administering to the patient an effective amount of ciforadenant, an effective amount of ipilimumab, and an effective amount of nivolumab, wherein a biological sample obtained from the patient has an increased level of gene expression of a biomarker gene relative to a control, wherein the biomarker gene is CXCL1, CXCL2, CXCL3, CXCL5, CXCL6, CXCL8, IL-1β, PTGS2, or a combination of two or more thereof. In embodiments, the effective amount of ciforadenant is about 200 mg per day, the effective amount of ipilimumab is about 1 mg/kg about once every three weeks, and the effective amount of nivolumab is about 3 mg/kg about once every three weeks. In embodiments, the effective amount of ciforadenant is about 100 mg twice per day, the effective amount of ipilimumab is about 1 mg/kg about once every three weeks, and the effective amount of nivolumab is about 3 mg/kg about once every three weeks. In embodiments, the biological sample is blood, tumor tissue, or tumor cells. In embodiments, the biological sample is tumor tissue or tumor cells. In embodiments, the biological sample is tumor tissue. In embodiments, the biological sample is tumor cells. In embodiments, the control is a population of healthy subjects (e.g., that do not have renal cell carcinoma). In embodiments, the effective amount of ipilimumab and/or the effective amount of nivolumab is a reduced effective amount.

In embodiments of the methods described herein, the biomarker gene is CXCL1, CXCL2, CXCL3, CXCL5, CXCL6, CXCL8, IL-1β, PTGS2, or a combination of two or more thereof. These genes are biomarkers of cells that are immunosuppressive and inhibit various T cell mediated immune responses to tumors. Immunosuppressive myeloid cells form in adenosine rich tumor microenvironments. The formation of these cells are blocked by inhibition of adenosine A2A receptors using adenosine A2A receptor antagonists.

In embodiments of the methods described herein, the biomarker gene comprises one biomarker gene selected from the group consisting of CXCL1, CXCL2, CXCL3, CXCL5, CXCL6, CXCL8, IL-1β, and PTGS2. In embodiments, the biomarker gene comprises at least one biomarker gene selected from the group consisting of CXCL1, CXCL2, CXCL3, CXCL5, CXCL6, CXCL8, IL-1β, and PTGS2. In embodiments, the biomarker gene comprises two biomarker genes selected from the group consisting of CXCL1, CXCL2, CXCL3, CXCL5, CXCL6, CXCL8, IL-1β, and PTGS2. In embodiments, the biomarker gene comprises at least two biomarker genes selected from the group consisting of CXCL1, CXCL2, CXCL3, CXCL5, CXCL6, CXCL8, IL-1β, and PTGS2. In embodiments, the biomarker gene comprises three biomarker genes selected from the group consisting of CXCL1, CXCL2, CXCL3, CXCL5, CXCL6, CXCL8, IL-1β, and PTGS2. In embodiments, the biomarker gene comprises at least three biomarker genes selected from the group consisting of CXCL1, CXCL2, CXCL3, CXCL5, CXCL6, CXCL8, IL-1β, and PTGS2. In embodiments, the biomarker gene comprises four biomarker genes selected from the group consisting of CXCL1, CXCL2, CXCL3, CXCL5, CXCL6, CXCL8, IL-1β, and PTGS2. In embodiments, the biomarker gene comprises at least four biomarker genes selected from the group consisting of CXCL1, CXCL2, CXCL3, CXCL5, CXCL6, CXCL8, IL-1β, and PTGS2. In embodiments, the biomarker gene comprises five biomarker genes selected from the group consisting of CXCL1, CXCL2, CXCL3, CXCL5, CXCL6, CXCL8, IL-1β, and PTGS2. In embodiments, the biomarker gene comprises at least five biomarker genes selected from the group consisting of CXCL1, CXCL2, CXCL3, CXCL5, CXCL6, CXCL8, IL-1β, and PTGS2. In embodiments, the biomarker gene comprises six biomarker genes selected from the group consisting of CXCL1, CXCL2, CXCL3, CXCL5, CXCL6, CXCL8, IL-1β, and PTGS2. In embodiments, the biomarker gene comprises at least six biomarker genes selected from the group consisting of CXCL1, CXCL2, CXCL3, CXCL5, CXCL6, CXCL8, IL-1β, and PTGS2. In embodiments, the biomarker gene comprises seven biomarker genes selected from the group consisting of CXCL1, CXCL2, CXCL3, CXCL5, CXCL6, CXCL8, IL-1β, and PTGS2. In embodiments, the biomarker gene comprises at least seven biomarker genes selected from the group consisting of CXCL1, CXCL2, CXCL3, CXCL5, CXCL6, CXCL8, IL-1B, and PTGS2.

In embodiments, the biomarker gene comprises CXCL1, CXCL2, CXCL3, CXCL5, CXCL6, CXCL8, IL-16, and PTGS2.

In embodiments, the biomarker gene consists of CXCL1, CXCL2, CXCL3, CXCL5, CXCL6, CXCL8, IL-1β, and PTGS2. With reference to this embodiment, “consists of” means that only the eight named biomarker genes (i.e., no other biomarker genes) are used in the methods described herein.

In embodiments of the methods described herein, the elevated level of gene expression is an elevated level of nucleic acid (e.g., RNA) expression or an elevated level of protein expression. Levels of gene expression can be determined by methods known in the art, such as those described herein. In embodiments, the elevated level of gene expression is an elevated level of protein expression. In embodiments, the level of the biomarkers is determined by an immunoassay, liquid chromatography-mass spectrometry (LC-MS), or a combination thereof. In embodiments, the elevated level of gene expression is an elevated level of nucleic acid expression. In embodiments, the elevated level of gene expression is an elevated level of RNA expression. In embodiments, the RNA is mRNA. In embodiments, RNA expression is detected by direct digital counting of nucleic acids, RNA sequencing (RNA-seq), quantitative reverse transcriptase polymerase chain reaction (RT-qPCR), quantitative polymerase chain reaction (qPCR), multiplex qPCR, microarray analysis, or a combination thereof. In embodiments, RNA expression is detected by RNA sequencing. RNA sequencing is a sequencing technique which uses next-generation sequencing (NGS) to reveal the presence and quantity of RNA in a biological sample. In embodiments, the gene expression level is an average of the gene expression level of the biomarker genes. In embodiments, the average of the gene expression level of the biomarker genes is an average of the normalized gene expression level of the biomarker genes. In embodiments, the gene expression level of the biomarker genes is a median of the gene expression level of the biomarker genes. In embodiments, the median of the gene expression level of the biomarker genes is a median of a normalized gene expression level of the biomarker genes.

Provided herein is a method for treating renal cell carcinoma in a patient in need thereof comprising: (a) detecting an increased level of myeloid suppressor cells, relative to a control, in a biological sample obtained from the patient; and (b) administering to the patient an effective amount of an adenosine A2A receptor antagonist and an effective amount of a CTLA-4 inhibitor. In embodiments, the adenosine A2A receptor antagonist is ciforadenant. In embodiments, the CTLA-4 inhibitor is ipilimumab. In embodiments, the method further comprises administering to the patient an effective amount of a PD-1 inhibitor. In embodiments, the PD-1 inhibitor is nivolumab. In embodiments, the biological sample is blood, tumor tissue, or tumor cells. In embodiments, the biological sample is blood. In embodiments, the biological sample is peripheral blood. In embodiments, the control is a population of healthy subjects (e.g., that do not have renal cell carcinoma).

Provided herein is a method for treating renal cell carcinoma in a patient in need thereof comprising: (a) detecting an increased level of myeloid suppressor cells, relative to a control, in a biological sample obtained from the patient; and (b) administering to the patient an effective amount of ciforadenant and an effective amount of ipilimumab. In embodiments, the effective amount of ciforadenant is about 200 mg per day and the effective amount of ipilimumab is about 1 mg/kg about once every three weeks. In embodiments, the effective amount of ciforadenant is about 100 mg twice per day and the effective amount of ipilimumab is about 1 mg/kg about once every three weeks. In embodiments, the biological sample is blood, tumor tissue, or tumor cells. In embodiments, the biological sample is blood. In embodiments, the biological sample is peripheral blood. In embodiments, the control is a population of healthy subjects (e.g., that do not have renal cell carcinoma).

Provided herein is a method for treating renal cell carcinoma in a patient in need thereof, the method comprising: (a) detecting an increased level of myeloid suppressor cells, relative to a control, in a biological sample obtained from the patient; and (b) administering to the patient an effective amount of ciforadenant, an effective amount of ipilimumab, and an effective amount of nivolumab. In embodiments, the effective amount of ciforadenant is about 200 mg per day, the effective amount of ipilimumab is about 1 mg/kg about once every three weeks, and the effective amount of nivolumab is about 3 mg/kg about once every three weeks. In embodiments, the effective amount of ciforadenant is about 100 mg twice per day, the effective amount of ipilimumab is about 1 mg/kg about once every three weeks, and the effective amount of nivolumab is about 3 mg/kg about once every three weeks. In embodiments, the biological sample is blood, tumor tissue, or tumor cells. In embodiments, the biological sample is blood. In embodiments, the biological sample is peripheral blood. In embodiments, the control is a population of healthy subjects (e.g., that do not have renal cell carcinoma). In embodiments, the effective amount of ipilimumab and/or the effective amount of nivolumab is a reduced effective amount.

Provided herein is a method for treating renal cell carcinoma in a patient in need thereof, the method comprising administering to the patient an effective amount of an adenosine A2A receptor antagonist and an effective amount of a CTLA-4 inhibitor; wherein a biological sample obtained from the patient has an increased level of myeloid suppressor cells. In embodiments, the adenosine A2A receptor antagonist is ciforadenant. In embodiments, the CTLA-4 inhibitor is ipilimumab. In embodiments, the method further comprises administering to the patient an effective amount of a PD-1 inhibitor. In embodiments, the PD-1 inhibitor is nivolumab. In embodiments, the biological sample is blood, tumor tissue, or tumor cells. In embodiments, the biological sample is blood. In embodiments, the biological sample is peripheral blood. In embodiments, the control is a population of healthy subjects (e.g., that do not have renal cell carcinoma).

Provided herein is a method for treating renal cell carcinoma in a patient in need thereof, the method comprising administering to the patient an effective amount of ciforadenant and an effective amount of ipilimumab, wherein a biological sample obtained from the patient has an increased level of myeloid suppressor cells relative to a control. In embodiments, the effective amount of ciforadenant is about 200 mg per day and the effective amount of ipilimumab is about 1 mg/kg about once every three weeks. In embodiments, the effective amount of ciforadenant is about 100 mg twice per day and the effective amount of ipilimumab is about 1 mg/kg about once every three weeks. In embodiments, the biological sample is blood, tumor tissue, or tumor cells. In embodiments, the biological sample is blood. In embodiments, the biological sample is peripheral blood. In embodiments, the control is a population of healthy subjects (e.g., that do not have renal cell carcinoma).

Provided herein is a method for treating renal cell carcinoma in a patient in need thereof, the method comprising administering to the patient an effective amount of ciforadenant, an effective amount of ipilimumab, and an effective amount of nivolumab, wherein a biological sample obtained from the patient has an increased level of myeloid suppressor cells relative to a control. In embodiments, the effective amount of ciforadenant is about 200 mg per day, the effective amount of ipilimumab is about 1 mg/kg about once every three weeks, and the effective amount of nivolumab is about 3 mg/kg about once every three weeks. In embodiments, the effective amount of ciforadenant is about 100 mg twice per day, the effective amount of ipilimumab is about 1 mg/kg about once every three weeks, and the effective amount of nivolumab is about 3 mg/kg about once every three weeks. In embodiments, the biological sample is blood, tumor tissue, or tumor cells. In embodiments, the biological sample is blood. In embodiments, the biological sample is peripheral blood. In embodiments, the control is a population of healthy subjects (e.g., that do not have renal cell carcinoma). In embodiments, the effective amount of ipilimumab and/or the effective amount of nivolumab is a reduced effective amount.

Provided herein is a method for treating renal cell carcinoma in a patient in need thereof comprising administering to the patient an effective amount of an adenosine A2A receptor antagonist, an effective amount of a CTLA-4 inhibitor, and an effective amount of a PD-1 inhibitor, wherein the effective amount of the CTLA-4 inhibitor and/or the PD-1 inhibitor is a reduced effective amount. In embodiments, the effective amount of the CTLA-4 inhibitor is a reduced effective amount. In embodiments, the effective amount of the PD-1 inhibitor is a reduced effective amount. In embodiments, the effective amount of the CTLA-4 inhibitor is a reduced effective amount and the effective amount of the PD-1 inhibitor is a reduced effective amount. In embodiments, the adenosine A2A receptor antagonist is ciforadenant. In embodiments, the CTLA-4 inhibitor is ipilimumab. In embodiments, the PD-1 inhibitor is nivolumab. In embodiments, a biological sample obtained from the patient has an increased level of expression of a biomarker gene relative to a control; wherein the biomarker gene comprises CXCL1, CXCL2, CXCL3, CXCL5, CXCL6, CXCL8, IL-1β, PTGS2, or a combination of two or more thereof. In embodiments, a biological sample obtained from the patient has an increased level of myeloid suppressor cells relative to a control.

Provided herein is a method for treating renal cell carcinoma in a patient in need thereof comprising administering to the patient an effective amount of ciforadenant, an effective amount of ipilimumab, and an effective amount of nivolumab, wherein the effective amount of ipilimumab and/or nivolumab is a reduced effective amount. In embodiments, the effective amount of ipilimumab is a reduced effective amount. In embodiments, the effective amount of nivolumab is a reduced effective amount. In embodiments, the effective amount of ipilimumab is a reduced effective amount and the effective amount of nivolumab is a reduced effective amount. In embodiments, a biological sample obtained from the patient has an increased level of expression of a biomarker gene relative to a control; wherein the biomarker gene comprises CXCL1, CXCL2, CXCL3, CXCL5, CXCL6, CXCL8, IL-1β, PTGS2, or a combination of two or more thereof. In embodiments, a biological sample obtained from the patient has an increased level of myeloid suppressor cells relative to a control.

In embodiments, the reduced effective amount of ipilimumab is less than 1 mg/kg every three weeks.

In embodiments, the reduced effective amount of ipilimumab is about 1 mg/kg once every four or more weeks. In embodiments, the reduced effective amount of ipilimumab is about 1 mg/kg once every four weeks. In embodiments, the reduced effective amount of ipilimumab is about 1 mg/kg once every five weeks. In embodiments, the reduced effective amount of ipilimumab is about 1 mg/kg once every six weeks. In embodiments, the reduced effective amount of ipilimumab is about 1 mg/kg once every seven weeks. In embodiments, the reduced effective amount of ipilimumab is about 1 mg/kg once every eight weeks. In embodiments, the reduced effective amount of ipilimumab is about 1 mg/kg once every four weeks to once every eight weeks.

In embodiments, the reduced effective amount of ipilimumab is about 0.9 mg/kg once every three weeks. In embodiments, the reduced effective amount of ipilimumab is about 0.8 mg/kg once every three weeks. In embodiments, the reduced effective amount of ipilimumab is about 0.7 mg/kg once every three weeks. In embodiments, the reduced effective amount of ipilimumab is about 0.6 mg/kg once every three weeks. In embodiments, the reduced effective amount of ipilimumab is about 0.5 mg/kg once every three weeks. In embodiments, the reduced effective amount of ipilimumab is about 0.4 mg/kg once every three weeks. In embodiments, the reduced effective amount of ipilimumab is about 0.3 mg/kg once every three weeks. In embodiments, the reduced effective amount of ipilimumab is about 0.2 mg/kg once every three weeks. In embodiments, the reduced effective amount of ipilimumab is about 0.1 mg/kg once every three weeks.

In embodiments, the reduced effective amount of ipilimumab is about 0.1 mg/kg to about 0.9 mg/kg once every three weeks. In embodiments, the reduced effective amount of ipilimumab is about 0.1 mg/kg to about 0.8 mg/kg once every three weeks. In embodiments, the reduced effective amount of ipilimumab is about 0.1 mg/kg to about 0.7 mg/kg once every three weeks. In embodiments, the reduced effective amount of ipilimumab is about 0.1 mg/kg to about 0.6 mg/kg once every three weeks. In embodiments, the reduced effective amount of ipilimumab is about 0.1 mg/kg to about 0.5 mg/kg once every three weeks. In embodiments, the reduced effective amount of ipilimumab is about 0.1 mg/kg to about 0.4 mg/kg once every three weeks. In embodiments, the reduced effective amount of ipilimumab is about 0.1 mg/kg to about 0.3 mg/kg once every three weeks. In embodiments, the reduced effective amount of ipilimumab is about 0.1 mg/kg to about 0.2 mg/kg once every three weeks.

In embodiments, the reduced effective amount of nivolumab is less than 3 mg/kg about once every three weeks.

In embodiments, the reduced effective amount of nivolumab is about 3 mg/kg once every four or more weeks. In embodiments, the reduced effective amount of nivolumab is about 3 mg/kg once every four weeks. In embodiments, the reduced effective amount of nivolumab is about 3 mg/kg once every five weeks. In embodiments, the reduced effective amount of nivolumab is about 3 mg/kg once every six weeks. In embodiments, the reduced effective amount of nivolumab is about 3 mg/kg once every seven weeks. In embodiments, the reduced effective amount of nivolumab is about 3 mg/kg once every eight weeks. In embodiments, the reduced effective amount of nivolumab is about 3 mg/kg once every four weeks to once every eight weeks.

In embodiments, the reduced effective amount of nivolumab is about 2.9 mg/kg about once every three weeks. In embodiments, the reduced effective amount of nivolumab is about 2.8 mg/kg about once every three weeks. In embodiments, the reduced effective amount of nivolumab is about 2.7 mg/kg about once every three weeks. In embodiments, the reduced effective amount of nivolumab is about 2.6 mg/kg about once every three weeks. In embodiments, the reduced effective amount of nivolumab is about 2.5 mg/kg about once every three weeks. In embodiments, the reduced effective amount of nivolumab is about 2.4 mg/kg about once every three weeks. In embodiments, the reduced effective amount of nivolumab is about 2.3 mg/kg about once every three weeks. In embodiments, the reduced effective amount of nivolumab is about 2.2 mg/kg about once every three weeks. In embodiments, the reduced effective amount of nivolumab is about 2.1 mg/kg about once every three weeks. In embodiments, the reduced effective amount of nivolumab is about 2.0 mg/kg about once every three weeks. In embodiments, the reduced effective amount of nivolumab is about 1.9 mg/kg about once every three weeks. In embodiments, the reduced effective amount of nivolumab is about 1.8 mg/kg about once every three weeks. In embodiments, the reduced effective amount of nivolumab is about 1.7 mg/kg about once every three weeks. In embodiments, the reduced effective amount of nivolumab is about 1.6 mg/kg about once every three weeks. In embodiments, the reduced effective amount of nivolumab is about 1.5 mg/kg about once every three weeks. In embodiments, the reduced effective amount of nivolumab is about 1.4 mg/kg about once every three weeks. In embodiments, the reduced effective amount of nivolumab is about 1.3 mg/kg about once every three weeks. In embodiments, the reduced effective amount of nivolumab is about 1.2 mg/kg about once every three weeks. In embodiments, the reduced effective amount of nivolumab is about 1.1 mg/kg about once every three weeks. In embodiments, the reduced effective amount of nivolumab is about 1.0 mg/kg about once every three weeks.

In embodiments, the reduced effective amount of nivolumab is about 1.0 mg/kg to about 2.9 mg/kg about once every three weeks. In embodiments, the reduced effective amount of nivolumab is about 1.0 mg/kg to about 2.8 mg/kg about once every three weeks. In embodiments, the reduced effective amount of nivolumab is about 1.0 mg/kg to about 2.7 mg/kg about once every three weeks. In embodiments, the reduced effective amount of nivolumab is about 1.0 mg/kg to about 2.6 mg/kg about once every three weeks. In embodiments, the reduced effective amount of nivolumab is about 1.0 mg/kg to about 2.5 mg/kg about once every three weeks. In embodiments, the reduced effective amount of nivolumab is about 1.0 mg/kg to about 2.4 mg/kg about once every three weeks. In embodiments, the reduced effective amount of nivolumab is about 1.0 mg/kg to about 2.3 mg/kg about once every three weeks. In embodiments, the reduced effective amount of nivolumab is about 1.0 mg/kg to about 2.2 mg/kg about once every three weeks. In embodiments, the reduced effective amount of nivolumab is about 1.0 mg/kg to about 2.1 mg/kg about once every three weeks. In embodiments, the reduced effective amount of nivolumab is about 1.0 mg/kg to about 2.0 mg/kg about once every three weeks.

### Kits and Assays

Provided here are kits comprising components, such as reagents and reaction mixtures, to conduct the assays to detect gene expression as described herein. As part of the kit, materials and instruction are provided, e.g., for storage and use of kit components. In embodiments, the kits comprise one or more of the following: a RNA probe that can hybridize to a RNA biomarker, pairs of primers that under appropriate reaction conditions can prime amplification of at least a portion of a RNA marker or a RNA encoding a polypeptide marker (e.g., by PCR), instructions on how to use the kit, and a label or insert indicating regulatory approval for diagnostic or therapeutic use. In embodiments, the kit further includes RNA microarrays comprising RNA of the disclosure or molecules which specifically bind to the RNA described herein. In embodiments, standard techniques of microarray technology are utilized to assess expression of the RNA. Polynucleotide arrays, particularly arrays that bind RNA described herein, also can be used for diagnostic applications, such as for identifying subjects that have a condition characterized by expression of polypeptide biomarkers.

“Assaying” or “detecting” means using an analytic procedure to qualitatively assess or quantitatively measure the presence or amount or the functional activity of a target entity (e.g., miRNA, mRNA). For example, detecting the level of RNA (such as miRNA or mRNA) means using an analytic procedure (such as an in vitro procedure) to qualitatively assess or quantitatively measure the presence or amount of the RNA. In embodiments, raw expression values are normalized by performing quantile normalization relative to the reference distribution and subsequent log 10-transformation. In embodiments, when RNA expression is detected using the nCounter& Analysis System marketed by Nanostring Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations).

The terms “probe” or “primer” refer to one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe or primers can be unlabeled or labeled as described below so that its binding to a target sequence can be detected (e.g., with a FRET donor or acceptor label). The probe or primer can be designed based on one or more particular (preselected) portions of a chromosome, e.g., one or more clones, an isolated whole chromosome or chromosome fragment, or a collection of polymerase chain reaction (PCR) amplification products. One of skill can adjust these factors to provide optimum hybridization and signal production for a given hybridization and detection procedures, and to provide the required resolution among different genes or genomic locations.

Probes and primers can also be immobilized on a solid surface (e.g., nitrocellulose, glass, quartz, fused silica slides), as in an array. Techniques for producing high density arrays can also be used for this purpose. One of skill will recognize that the precise sequence of particular probes and primers can be modified from the target sequence to a certain degree to produce probes that are “substantially identical” or “substantially complementary to” a target sequence, but retain the ability to specifically bind to (i.e., hybridize specifically to) the same targets from which they were derived.

The term “capable of hybridizing to” refers to a polynucleotide sequence that forms Watson-Crick bonds with a complementary sequence. One of skill will understand that the percent complementarity need not be 100% for hybridization to occur, depending on the length of the polynucleotides, length of the complementary region(e.g. 5, 10, 15, 20, 25, 30, 35, 40, 45, 50, 60, 70, 80, 90, 100, or more bases in length), and stringency of the conditions. For example, a polynucleotide (e.g., primer or probe) can be capable of binding to a polynucleotide having 60%, 65%, 70%, 75%, 80%, 85%, 90%, 91%, 92%, 93%, 94%, 95%, 96%, 97%, 98%, 99% or 100% complementarity over the stretch of the complementary region.

In embodiments, methods include detecting a level of a biomarker with a specific binding agent (e.g., an agent that binds to a protein or nucleic acid molecule). Exemplary binding agents include an antibody or a fragment thereof, a detectable protein or a fragment thereof, a nucleic acid molecule such as an oligonucleotide/polynucleotide comprising a sequence that is complementary to patient genomic DNA, mRNA or a cDNA produced from patient mRNA, or any combination thereof. In embodiments, an antibody is labeled with detectable moiety, e.g., a fluorescent compound, an enzyme or functional fragment thereof, or a radioactive agent. In embodiments, an antibody is detectably labeled by coupling it to a chemiluminescent compound. In embodiments, the presence of the chemiluminescent-tagged antibody is then determined by detecting the presence of luminescence that arises during the course of chemical reaction. Non-limiting examples of useful chemiluminescent labeling compounds are luminol, isoluminol, theromatic acridinium ester, imidazole, acridinium salt and oxalate ester.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomarkers present in the sample do not significantly bind to the binding agent specific for the target molecule. In embodiments, the level of binding to a biomolecule other than the target biomarker results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10⁻⁷-10⁻⁹), with high affinity antibodies in the low nanomolar (10⁻⁹) or pico molar (10⁻¹²) range for its specific target biomarker.

In embodiments, the subject matter provides a composition comprising a binding agent, wherein the binding agent is attached to a solid support, (e.g., a strip, a polymer, a bead, a nanoparticle, a plate such as a multiwell plate, or an array such as a microarray). In embodiments relating to the use of a nucleic acid probe attached to a solid support (such as a microarray), a nucleic acid in a test sample may be amplified (e.g., using PCR) before or after the nucleic acid to be measured is hybridized with the probe. In embodiments, reverse transcription polymerase chain reaction (RT-PCR) is used to detect mRNA levels. In embodiments, a probe on a solid support is used, and mRNA (or a portion thereof) in a biological sample is converted to cDNA or partial cDNA and then the cDNA or partial cDNA is hybridized to a probe (e.g., on a microarray), hybridized to a probe and then amplified, or amplified and then hybridized to a probe. In embodiments, a strip may be a nucleic acid-probe coated porous or non-porous solid support strip comprising linking a nucleic acid probe to a carrier to prepare a conjugate and immobilizing the conjugate on a porous solid support. In embodiments, the support or carrier comprises glass, polystyrene, polypropylene, polyethylene, dextran, nylon, amylases, natural and modified celluloses, polyacrylamides, gabbros, and magnetite. In embodiments, the nature of the carrier can be either soluble to some extent or insoluble for the purposes of the present subject matter. In embodiments, the support material may have any structural configuration so long as the coupled molecule is capable of binding to a binding agent (e.g., an antibody). In embodiments, the support configuration may be spherical, as in a bead, or cylindrical, as in the inside surface of a test tube, or the external surface of a rod. In embodiments, the surface may be flat such as a plate (or a well within a multiwell plate), sheet, test strip, polystyrene beads. Those skilled in the art will know many other suitable carriers for binding antibody or antigen, or will be able to ascertain the same by use of routine experimentation.

In embodiments, a solid support comprises a polymer, to which an agent is chemically bound, immobilized, dispersed, or associated. In embodiments, a polymer support may be, e.g., a network of polymers, and may be prepared in bead form (e.g., by suspension polymerization). In embodiments, the location of active sites introduced into a polymer support depends on the type of polymer support. In embodiments, in a swollen-gel-bead polymer support the active sites are distributed uniformly throughout the beads, whereas in a macroporous-bead polymer support they are predominantly on the internal surfaces of the macropores. In embodiments, the solid support, e.g., a device, may contain a biomarker binding agent alone or together with a binding agent for at least one, two, three or more other biomarkers.

In embodiments, detection is accomplished using an ELISA or Western blot format. In embodiments, the binding agent comprises an nucleic acid (e.g., a probe or primers that are complementary for mRNA or cDNA), and the detecting step is accomplished using a polymerase chain reaction (PCR) or Northern blot format, or other means of detection. In embodiments, a probe or primer is about 10-20, 15-25, 15-35, 15-25, 20-80, 50-100, or 10-100 nucleotides in length, e.g., about 10, 12, 15, 20, 25, 30, 35, 40, 45, 50, 55, 60, 70, 80, 90, or 100 nucleotides in length or less than about 30, 35, 40, 45, 50, 55, 60, 70, 80, 90, or 100 nucleotides in length.

In embodiments, the cells in a biological sample are lysed to release a protein or nucleic acid. Numerous methods for lysing cells and assessing protein and nucleic acid levels are known in the art. In embodiments, cells are physically lysed, such as by mechanical disruption, liquid homogenization, high frequency sound waves, freeze/thaw cycles, with a detergent, or manual grinding. Non-limiting examples of detergents include Tween 20, Triton X-100, and sodium dodecyl sulfate (SDS). Non-limiting examples of assays for determining the level of a protein include HPLC, LC/MS, ELISA, immunoelectrophoresis, Western blot, immunohistochemistry, and radioimmunoassays. Non-limiting examples of assays for determining the level of an mRNA include Northern blotting, RT-PCR, RNA sequencing, and qRT-PCR.

In embodiments, once a suitable biological sample has been obtained, it is analyzed to quantitate the expression level of each of the biomarker genes. In embodiments, determining the expression level of a gene comprises detecting and quantifying RNA transcribed from that gene or a protein translated from such RNA. In embodiments, the RNA includes mRNA transcribed from the gene, and/or specific spliced variants thereof and/or fragments of such mRNA and spliced variants.

In embodiments, raw expression values are normalized by performing quantile normalization relative to the reference distribution and subsequent log 10-transformation. In embodiments, when the gene expression is detected using the nCounter® Analysis System marketed by NanoString® Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations). In embodiments, the T-effector signature score is then calculated as the arithmetic mean of normalized values for each of the genes in the gene signature.

In embodiments, oligonucleotides in kits are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

A “detectable agent” or “detectable moiety” is a compound or composition detectable by appropriate means such as spectroscopic, photochemical, biochemical, immunochemical, chemical, magnetic resonance imaging, or other physical means. The RNA described herein and the expression level of the RNA described herein may be accomplished through the use of a detectable moiety in an assay or kit. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, magnetic beads, paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides, fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles, iodinated contrast agents, barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

### Embodiments 1-24

Embodiment 1. A method for treating renal cell carcinoma in a patient in need thereof, the method comprising: (a) detecting an increased level of gene expression relative to a control of a biomarker gene in a biological sample obtained from the patient, wherein the biomarker gene is CXCL1, CXCL2, CXCL3, CXCL5, CXCL6, CXCL8, IL-1β, PTGS2, or a combination of two or more thereof; and (b) administering to the patient an effective amount of an adenosine A2A receptor antagonist and an effective amount of a CTLA-4 inhibitor.

Embodiment 2. A method for treating renal cell carcinoma in a patient in need thereof, the method comprising administering to the patient an effective amount of an adenosine A2A receptor antagonist and an effective amount of a CTLA-4 inhibitor; wherein a biological sample obtained from the patient has an increased level of gene expression relative to a control of a biomarker gene, wherein the biomarker gene is CXCL1, CXCL2, CXCL3, CXCL5, CXCL6, CXCL8, IL-1β, PTGS2, or a combination of two or more thereof.

Embodiment 3. The method of Embodiment 1 or 2, wherein the biomarker gene comprises CXCL1, CXCL2, CXCL3, CXCL5, CXCL6, CXCL8, IL-1β, and PTGS2.

Embodiment 4. The method of Embodiment 1 or 2, wherein the biomarker gene consists of CXCL1, CXCL2, CXCL3, CXCL5, CXCL6, CXCL8, IL-1β, and PTGS2.

Embodiment 5. The method of any one of Embodiments 1 to 4, wherein the biological sample is a tumor sample.

Embodiment 6. The method of any one of Embodiments 1 to 4, wherein the biological sample is tumor cells.

Embodiment 7. A method for treating renal cell carcinoma in a patient in need thereof, the method comprising: (a) detecting an increased level of myeloid suppressor cells relative to a control in a biological sample obtained from the patient; and (b) administering to the patient an effective amount of an adenosine A2A receptor antagonist and an effective amount of a CTLA-4 inhibitor.

Embodiment 8. A method for treating renal cell carcinoma in a patient in need thereof, the method comprising administering to the patient an effective amount of an adenosine A2A receptor antagonist and a CTLA-4 inhibitor; wherein a biological sample obtained from the patient has an increased level of myeloid suppressor cells relative to a control.

Embodiment 9. The method of Embodiment 7 or 8, wherein the biological sample is blood.

Embodiment 10. The method of Embodiment 7 or 8, wherein the biological sample is peripheral blood.

Embodiment 11. The method of any one of Embodiments 1 to 10, wherein the adenosine A2A receptor antagonist is ciforadenant.

Embodiment 12. The method of Embodiment 11, wherein the effective amount of ciforadenant is 100 mg twice per day.

Embodiment 13. The method of any one of Embodiments 1 to 12, wherein the CTLA-4 inhibitor is ipilimumab.

Embodiment 14. The method of Embodiment 13, wherein the effective amount of ipilimumab is about 1 mg/kg about once every three weeks.

Embodiment 15. The method of any one of Embodiments 1 to 14, further comprising administering to the patient an effective amount of a PD-1 inhibitor.

Embodiment 16. The method of Embodiment 15, wherein the PD-1 inhibitor is nivolumab.

Embodiment 17. The method of Embodiment 16, wherein the effective amount of nivolumab is about 3 mg/kg about once every three weeks.

Embodiment 18. A method for treating renal cell carcinoma in a patient in need thereof, the method comprising administering to the patient an effective amount of an adenosine A2A receptor antagonist, an effective amount of a CTLA-4 inhibitor, and an effective amount of a PD-1 inhibitor, wherein the effective amount of the CTLA-4 inhibitor and/or the PD-1 inhibitor is a reduced effective amount.

Embodiment 19. The method of Embodiment 18, wherein the adenosine A2A receptor antagonist is ciforadenant, the CTLA-4 inhibitor is ipilimumab, and the PD-1 inhibitor is nivolumab.

Embodiment 20. The method of Embodiment 19, wherein the reduced effective amount of ipilimumab is less than 1 mg/kg every three weeks.

Embodiment 21. The method of Embodiment 19 or 20, wherein the reduced effective amount of nivolumab is less than 3 mg/kg every three weeks.

Embodiment 22. The method of any one of Embodiments 18 to 21, wherein the effective amount of ciforadenant is 100 mg twice daily.

Embodiment 23. The method of any one of Embodiments 18 to 22, wherein a biological sample obtained from the patient has an increased level of expression of a biomarker gene relative to a control; wherein the biomarker gene is CXCL1, CXCL2, CXCL3, CXCL5, CXCL6, CXCL8, IL-1β, PTGS2, or a combination of two or more thereof.

Embodiment 24. The method of any one of Embodiments 18 to 23, wherein a biological sample obtained from the patient has an increased level of myeloid suppressor cells relative to a control.

1-24. (canceled)

25. A method for treating renal cell carcinoma in a patient in need thereof, the method comprising:
(a) detecting:
(i) an increased level of gene expression of a biomarker gene, relative to a control, in a biological sample obtained from the patient, wherein the biomarker gene is CXCL1, CXCL2, CXCL3, CXCL5, CXCL6, CXCL8, IL-1β, PTGS2, or a combination of two or more thereof, or
(ii) an increased level of myeloid suppressor cells relative to a control in a biological sample obtained from the patient; and
(b) administering to the patient an effective amount of an adenosine A2A receptor and an effective amount of a CTLA-4 inhibitor.

26. The method of claim 25, wherein the biomarker gene comprises CXCL1, CXCL2, CXCL3, CXCL5, CXCL6, CXCL8, IL-1β, and PTGS2.

27. The method of claim 25, wherein the biomarker gene consists of CXCL1, CXCL2, CXCL3, CXCL5, CXCL6, CXCL8, IL-1β, and PTGS2.

28. The method of claim 25, wherein the adenosine A2A receptor antagonist is ciforadenant.

29. The method of claim 28, wherein the effective amount of ciforadenant is about 100 mg twice per day.

30. The method of claim 25, wherein the CTLA-4 inhibitor is ipilimumab.

31. The method of claim 30, wherein the effective amount of ipilimumab is about 1 mg/kg about once every three weeks.

32. The method of claim 25, further comprising administering to the patient an effective amount of a PD-1 inhibitor.

33. The method of claim 32, wherein the PD-1 inhibitor is nivolumab.

34. The method of claim 33, wherein the effective amount of nivolumab is about 3 mg/kg about once every three weeks.

35. The method of claim 25, wherein the biological sample is a tumor sample or tumor cells.

36. The method of claim 25, wherein the biological sample is blood.

37. The method of claim 25, wherein the biological sample is peripheral blood.

38. A method for treating renal cell carcinoma in a patient in need thereof, the method comprising administering to the patient an effective amount of an adenosine A2A receptor antagonist and an effective amount of a CTLA-4 inhibitor; wherein a biological sample obtained from the patient has:
(i) an increased level of gene expression relative to a control of a biomarker gene, wherein the biomarker gene is CXCL1, CXCL2, CXCL3, CXCL5, CXCL6, CXCL8, IL-1β, PTGS2, or a combination of two or more thereof, or
(ii) an increased level of myeloid suppressor cells relative to a control.

39. The method of claim 38, wherein the adenosine A2A receptor antagonist is ciforadenant and the the CTLA-4 inhibitor is ipilimumab.

40. The method of claim 39, wherein the effective amount of ciforadenant is about 100 mg twice per day and the effective amount of ipilimumab is about 1 mg/kg about once every three weeks.

41. The method of claim 38, further comprising administering to the patient an effective amount of nivolumab.

42. The method of claim 41, wherein the effective amount of nivolumab is about 3 mg/kg about once every three weeks.

43. A method for treating renal cell carcinoma in a patient in need thereof, the method comprising administering to the patient an effective amount of an adenosine A2A receptor antagonist, an effective amount of a CTLA-4 inhibitor, and an effective amount of a PD-1 inhibitor, wherein: (a) the effective amount of the CTLA-4 inhibitor is a reduced effective amount; (b) the effective amount of the PD-1 inhibitor is a reduced effective amount; or (c) the effective amount of the CTLA-4 inhibitor is a reduced effective amount and the effective amount of the PD-1 inhibitor is a reduced effective amount.

44. The method of claim 43, wherein the adenosine A2A receptor antagonist is ciforadenant, the CTLA-4 inhibitor is ipilimumab, and the PD-1 inhibitor is nivolumab.
